(19) United States
(12) Patent Application Publication
(10) Pub. No.: US 2022/0397344 A1
(43) Pub. Date: Dec. 15, 2022

(54) COMBINED PLANT FOR CRYOGENIC SEPARATION AND LIQUEFACTION OF METHANE AND CARBON DIOXIDE COMPRISED IN A BIOGAS STREAM

(71) Applicant: L'Air Liquide Société Anonyme pour I'Etude et I'Exploitation des Procédés Procédés Georges Claude, Paris (FR)

(72) Inventor: Jorge Ernesto Tovar Ramos, Cachan (FR)

(21) Appl. No.: 17/835,622

(22) Filed: Jun. 8, 2022

(30) Foreign Application Priority Data
Jun. 9, 2021 (FR) 2106082

(51) Int. Cl. F25J 3/02; C01B 32/50; C07C 7/00

(52) U.S. Cl. CPC F25J 3/0233; C01B 32/50; C07C 7/00; F25J 3/0223; F25J 3/0266; C01B 2210/0009; C01B 2210/007

(57) ABSTRACT

A combined plant for cryogenic separation and liquefaction of methane and carbon dioxide in a biogas stream, including a mixing means, a compressor, a first exchanger, a distillation column, a second exchanger, a separating means, an expanding means, and a separator vessel. Wherein the mixing means is configured such that the recycle gas is the overhead vapour stream, and the first exchanger and the expanding means are combined.

### CROSS REFERENCE TO RELATED APPLICATIONS

This application claims the benefit of priority under 35 U.S.C. § 119 (a) and (b) to French Patent Application No. 2106082, filed Jun. 9, 2021, the entire contents of which are incorporated herein by reference.

### BACKGROUND

The present invention relates to a plant and a process for producing liquid methane and liquid carbon dioxide from a biogas stream.

Biogas is the gas produced during the degradation of organic matter in the absence of oxygen (anaerobic fermentation), also known as methanization. This may be natural degradation—it is thus observed in marshland or in household waste landfills—but the production of biogas may also result from the methanization of waste in a dedicated reactor referred to as a methanizer or digester.

By virtue of its main constituents—methane and carbon dioxide—biogas is a powerful greenhouse gas; at the same time, it also constitutes a source of renewable energy that is appreciable in the context of the increasing scarcity of fossil fuels.

Biogas predominantly contains methane (CH₄) and carbon dioxide (CO₂), in proportions which can vary according to the way in which it is obtained, but also contains, in smaller proportions, water, nitrogen, hydrogen sulfide, oxygen, and also other organic compounds, in trace amounts.

Depending on the organic matter that has been degraded and on the techniques used, the proportions of the components differ, although on average biogas includes, on a dry gas basis, from 30% to 75% methane, from 15% to 60% CO₂, from 0 to 15% nitrogen, from 0 to 5% oxygen and trace compounds.

After a step of pretreating these contaminants, the biogas can be used as is to supply a boiler or a cogeneration unit, or else purified to obtain a gas which meets the specifications for injection into the natural gas network (e.g.: 3% CO₂ max).

In numerous regions of Europe and throughout the world, the natural gas network is not always accessible close to the areas of production of fermentable waste. Furthermore, while there is no need for heat at the biogas production site, depending on the purchase price of electricity, cogeneration does not always have a sufficient output to render profitable the major investment in a digestion unit. It is then advantageous in these two cases to transport the biogas to a distribution or consumption point. The liquefaction of biogas after purification would make it possible to transport biomethane at a lower cost. According to the regulations in certain geographic zones, it is forbidden to release CH₄ into the environment; this adds an additional constraint and restricts the choice of biogas separation processes to highly effective methods.

Today, biogas purification processes are mainly based on absorption, permeation or adsorption techniques. These systems then require the addition of a supplementary module in order to obtain biomethane in the liquid form. Moreover, in the majority of cases, the content of CO₂ in the biogas at the end of this purification step is still too high to supply such liquefaction systems.

A system of cryotrapping based on the principles of reversible exchangers has been proposed. This system is based on the solidification of the CO₂ present in the biogas on a cold surface (trapping), followed by a step of sublimation or liquefaction of the CO₂ using a hot source. For a continuous production of biomethane, is is then necessary to work with several exchangers in parallel. Their solution makes it possible to separate and liquefy the methane and the CO₂ in two separate steps, but it is not possible to recover the cold used in the solidification of the CO₂.

Starting from there, one problem that arises is that of providing a method of separating and liquefying methane and CO₂ from biogas with a minimum loss of methane and using a minimum number of operations.

### SUMMARY

One solution of the present invention is a combined plant for cryogenic separation and liquefaction of methane and carbon dioxide comprised in a biogas stream, comprising:
a means M1 for mixing the biogas 1 with a recycle gas R,
a compressor for compressing the mixture to the distillation pressure,
an exchanger E01 for cooling the compressed mixture,
a distillation column K01 supplied with the cooled mixture and making it possible to produce methane at the top of the column and a CO₂-enriched liquid at the bottom of the column,
an exchanger E02 for liquefying the methane produced at the top of the column,
a means M2 for separating the liquefied methane into two portions: a “reflux” portion 3 and a “product” portion 2,
a means M3 for expanding and heating the CO₂-enriched liquid recovered at the bottom of the column and for recovering the cold from the CO₂-enriched liquid, and
a separator vessel V01 for receiving the CO₂-enriched stream from the means M3 and for recovering an overhead vapour and liquid CO₂ 4,

with
the means M1 such that the recycle gas R corresponds to the overhead vapour recovered at the outlet of the separator vessel V01, and
the exchanger E01 and the means M3 being combined.

Depending on the case, the plant according to the invention may have one or more of the following characteristics:
the plant comprises, upstream of the means M1, means for drying and desulfurization of the biogas;
the plant comprises, upstream of the means M1, a means C01 for compressing the biogas to the pressure of the recycle gas R;
the plant comprises, upstream of the means M1, a means C01E and/or CO₂E for cooling the biogas to ambient temperature;
the exchanger E02 is within a closed refrigeration circuit;
the refrigeration circuit uses methane as refrigerant fluid;
the distillation column K01 comprises heating at the bottom of the column.

The present invention also relates to a combined process of cryogenic separation and liquefaction of methane and carbon dioxide comprised in a biogas stream, using the plant as defined previously, and comprising:
a) a step of mixing the biogas 1 with a recycle gas R,
b) a step of compressing the mixture to the distillation pressure,
c) a step of cooling the compressed mixture in the exchanger E01,
d) a step of distilling the cooled mixture in the distillation column K01 so as to produce methane at the top of the column and a CO₂-enriched liquid at the bottom of the column,
e) a step of liquefying the methane produced at the top of the column in the exchanger E02,
f) a separation step for separating the liquefied methane into two portions: a “reflux” portion 3 and a “product” portion 2,
g) a step of expanding and heating the CO₂-enriched liquid recovered at the bottom of the column in the exchanger E01, and of recovering the cold from the CO₂-enriched liquid, and
h) a step of separating the CO₂-enriched stream resulting from the exchanger E01 in the separator vessel V01 into liquid CO₂ 4 and overhead vapour,

with the recycle gas R corresponding to the overhead vapour produced in step a).

Depending on the case, the process according to the invention may have one or more of the features below:
The process comprises, upstream of step a), drying and desulfurization steps.
The process comprises, upstream of step a step of compressing the biogas to the pressure of the recycle gas R.
The process comprises, upstream of step a), a step of cooling the biogas to ambient temperature.
The process comprises, downstream of step h), a step of heating the liquid CO₂ so as to vaporize it.
Step e) is performed by cooling the produced methane by means of a refrigerant fluid. The product at the top of the column is pure methane vapour. This vapour is liquefied in an exchanger cooled by a closed refrigeration cycle thermally integrated with the process. A portion of the liquid methane leaves the circuit as product and another portion is used as column reflux.
In step b), the mixture is compressed to a pressure of between 7 and 46 bar.

The process according to the invention makes it possible to separate and liquefy the products of the biogas in a single combined distillation/liquefaction operation. The operating conditions of the products at the inlet and outlet of the column and in the recycle section have been calculated to prevent the formation of solid CO₂.

The thermal integration between the streams of the separation section and those of the refrigeration cycle enable the recovery of the cold used in the liquefaction of the CO₂ and in the recycling of the liquid methane. It is possible to completely or partly recover the energy used in the liquefaction of the CO₂ if this CO₂ is not desired as a product or when it can be used in the gaseous state.

### BRIEF DESCRIPTION OF THE DRAWING

For a further understanding of the nature and objects for the present invention, reference should be made to the following detailed description, taken in conjunction with the accompanying drawings, in which like elements are given the same or analogous reference numbers and wherein:

FIG. 1 illustrates a refrigeration circuit in accordance with one embodiment of the present invention.

### DETAILED DESCRIPTION OF PREFERRED EMBODIMENTS

The pretreated biogas 1 (pretreated by drying, desulfurization) is introduced into the process at atmospheric pressure and temperature, it is compressed a first time in a compressor C01, to the pressure of the recycle circuit (around 8 bar). After compression, it is cooled in C01E to ambient temperature with CW (=Cooling Water) or air.

Next, it is mixed with a recycle stream R, the mixture is compressed in a compressor CO₂, to the pressure of the distillation column (around 15 bar) or more depending on the requirements of the downstream exchanger E01 and it is cooled to ambient temperature in CO₂E, with CW or air.

Preferably, C01E and CO₂E are shell and tube exchangers (cooler of the compressors).

The mixture of biogas—recycle stream R is sent to the exchanger E01. The main purpose of this exchanger is to cool the mixture in preparation for the distillation. The mixture can then be expanded or supplied directly to the column where it will be used as reboiler.

If there is no heat source at the bottom of the column, it is necessary to inject the mixture into the bottom to ensure the circulation of vapour from the bottom. If there is a heat source in the bottom of the column (reboiler), the mixture is introduced higher up in the column.

The distillation column K01 separates the methane from the carbon dioxide. The feed for the column is the biogas+recycle stream R mixture. This feed acts as main reboiler; an additional source of heat may also be used (for example an electrical resistance heater, vapour or a portion of the hot biogas in indirect contact). The product at the top of the column is pure CH₄ in the vapour state. The bottom product is a liquid rich in CO₂, containing around 95%-98%.

The methane at the top of the column is liquefied in the exchanger E02, against a fluid from a closed refrigeration circuit. A portion of the methane leaves the cycle as product 2 and the other portion 3 (reflux portion) is used as recycle for the column and reinjected into the top of the column.

The CO₂-enriched liquid recovered at the bottom of the column is expanded and heated in the exchanger E01 countercurrent to the biogas—recycle stream R mixture.

The CO₂-enriched stream from the exchanger E01 is sent to the separator vessel V01.

The overhead vapour of the vessel V01 is reheated in the exchanger E01 and then mixed with the biogas. It corresponds to the stream previously named “recycle stream R”.

The liquid from the bottom of the vessel V01 is the pure CO₂ 4. This can, depending on the requirements, leave the process as product or be reheated in the exchanger E01 and in another exchanger E03 of the refrigeration circuit in order to be completely vaporized before leaving the cycle. Note that the pure CO₂ could alternatively be reheated and vaporised in the exchanger E03 without passing through the exchanger E01.

The exchanger E01 therefore uses, as sources of cold: the CO₂-enriched liquid recovered at the bottom of the column, the overhead vapour from the vessel V01 named “recycle stream R” at the outlet of the exchanger E01, and optionally the pure liquid CO₂ recovered at the bottom of the vessel V01 in the case where the vaporisation thereof is desired.

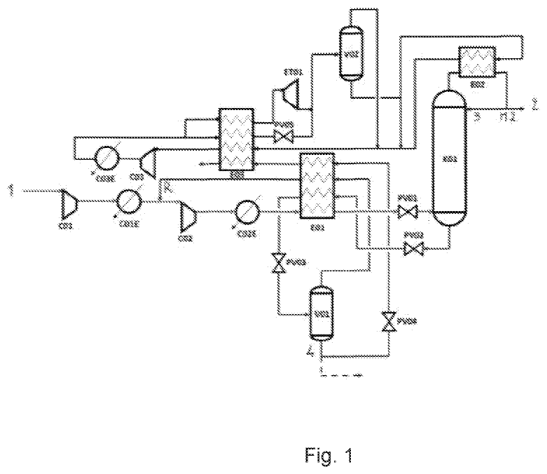

The process requires an input of refrigeration power in order to operate. This input of cold is represented in FIG. 1 by the refrigeration circuit: it is composed of:
a compressor C03 with cooler C03E;
an exchanger E03 which cools the compressed fluid using the recycled refrigerant fluid and the cold recovered from the separation cycle;
a turbine ET01 and a JT valve PV05, for the expansion of the refrigerant fluid and production of cold;
a separator vessel V02 separating the vapour and liquid phases of the refrigerant fluid;
an exchanger E02 which uses the liquid phase of the refrigerant fluid to liquefy the biomethane at the top of the distillation column.
The refrigerant fluid used in the scheme is CH₄ but it can be replaced by other fluids such as N₂, N₂+H₂, inter alia.

This refrigeration cycle can be replaced by other sources of cold (depending on the amount of liquid biomethane to be produced). By way of example, but not exclusively:
using a source of liquid nitrogen;
by a Brayton cycle process.

It will be understood that many additional changes in the details, materials, steps and arrangement of parts, which have been herein described in order to explain the nature of the invention, may be made by those skilled in the art within the principle and scope of the invention as expressed in the appended claims. Thus, the present invention is not intended to be limited to the specific embodiments in the examples given above.

What is claimed is:

1. A combined plant for cryogenic separation and liquefaction of methane and carbon dioxide in a biogas stream, comprising:
a mixing means for mixing a biogas stream with a recycle gas stream, thereby producing a mixed biogas stream,
a compressor for compressing the mixed biogas stream to ae pressure suitable for distillation, thereby producing a compressed stream,
a first exchanger for cooling the compressed stream, thereby producing a cooled stream,
a distillation column, comprising a top and a bottom, and configured to be supplied with the cooled stream and configured to produce a methane stream at the top and a CO₂-enriched liquid stream at the bottom,
a second exchanger for liquefying the methane stream, thereby producing a liquefied methane stream,
a separating means for separating the liquefied methane stream into two portions: a “reflux” portion and a “product” portion,
an expanding means for expanding and heating the CO₂-enriched liquid stream and for recovering cold from the CO₂-enriched liquid stream, thereby producing a heated CO₂-enriched stream, and
a separator vessel for receiving the CO₂-enriched stream and for recovering an overhead vapour stream and liquid CO₂ stream, wherein
the mixing means is configured such that the recycle gas is the overhead vapour stream and
the first exchanger and the expanding means are combined.

2. The plant according to claim 1, further comprising, upstream of the mixing means, a drying means for drying and desulfurization of the biogas stream.

3. The plant according to claim 1, further comprising, upstream of the mixing means, a compressing means for compressing the biogas stream to the pressure of the recycle gas stream.

4. The plant according to claim 1, further comprising, upstream of the mixing means, a cooling means for cooling the biogas stream to ambient temperature.

5. The plant according to claim 1, wherein the second exchanger is within a closed refrigeration circuit.

6. The plant according to claim 5, wherein the refrigeration circuit uses methane as refrigerant fluid.

7. The plant according to claim 1, wherein the distillation column comprises heating at the bottom.

8. A combined process of cryogenic separation and liquefaction of methane and carbon dioxide in a biogas stream, using the plant as defined in claim 1, and the process comprising:
a) mixing the biogas stream with the recycle gas stream,
b) compressing the mixed biogas stream to the distillation pressure,
c) cooling the compressed stream in the first exchanger,
d) distilling the cooled stream in the distillation column, thereby producing the methane stream and the CO₂-enriched liquid stream,
e) liquefying the methane stream in the second exchanger,
f) separating the liquefied methane stream into the “reflux” portion and the “product” portion,
g) expanding and heating the CO₂-enriched liquid stream in the first exchanger, and recovering the cold from the CO₂-enriched liquid stream, and
h) separating the heated CO₂-enriched stream in the separator vessel V01 into the liquid CO₂ stream and the overhead vapour stream,
with the recycle gas stream is the overhead vapour produced in step a).

9. The process according to claim 8, further comprising, upstream of step a), drying and of desulfurization the biogas stream.

10. The process according to claim 8 further comprising, upstream of step a), a step of compressing the biogas stream to the pressure of the recycle gas stream.

11. The process according to claim 8, further comprising, upstream of step a), a step of cooling the biogas stream to ambient temperature.

12. The process according to claim 8, further comprising, downstream of step h), a step of heating and vaporizing the liquid CO₂ stream.

13. The process according to claim 8, wherein step e) is performed by cooling the methane stream by means of a refrigerant fluid.

14. The process according to claim 8, wherein, in step b), the mixed biogas stream is compressed to a pressure of between 7 and 46 bar.
